(19) United States
(12) Patent Application Publication
(10) Pub. No.: US 2016/0213432 A1
(43) Pub. Date: Jul. 28, 2016

(54) HUB DESIGN AND METHODS FOR OPTICAL SHAPE SENSING REGISTRATION

(71) Applicant: KONINKLIJKE PHILIPS N.V., Eindhoven (NL)

(72) Inventors: MOLLY LARA FLEXMAN, MELROSE, MA (US); MARISSA PATRICIA DREYER, KETCHUM, ID (US); MARCO VERSTEGE, EINDHOVEN (NL); DAVID PAUL NOONAN, NEW YORK, NY (US)

(21) Appl. No.: 15/025,900

(22) PCT Filed: Sep. 19, 2014
(86) PCT No.: PCT/IB2014/064647

§ 371 Date: Mar. 30, 2016

Related U.S. Application Data
(60) Provisional application No. 61/885,527, filed on Oct. 2, 2013.

(51) Int. Cl. A61B 34/20

(52) U.S. Cl. CPC A61B 34/20; A61B 2034/2061; A61B 2034/2065; A61B 2017/00477

(57) ABSTRACT

An optical shape sensing hub includes a longitudinal body (210) forming a cavity configured to receive two or more optical shape sensing (OSS) enabled instruments. One or more mechanical features (212, 214) are disposed within the cavity or on the longitudinal body to maintain the two or more OSS enabled instruments in a fixed geometrical configuration relative to one another such that distally to the longitudinal body the two or more OSS enabled instruments have shape sensed reconstruction data registered therebetween. The disclosed hub can be used in a shape sensing system to determine shapes of OSS enabled instruments. It is further disclosed a method for registering two or more OSS enabled instruments by generating a hub template of an expected shape of the hub in OSS data, searching measured OSS data to match the hub template to determine a hub position in the OSS data and finding overlap in the OSS data relative to the hub position.

### BACKGROUND

1. Technical Field

This disclosure relates to medical instruments and more particularly to shape sensing optical fiber registration tools and methods for use.

2. Description of the Related Art

Optical shape sensing (OSS) uses a multi-core optical fiber to reconstruct shape along the length of a device. A position along the sensor, known as z=0, provides a starting point for shape reconstruction in space. In most applications, this reconstructed shape is then overlaid with either a pre-operative image (using e.g., computed tomography (CT), magnetic resonance imaging (MRI), fluoroscopy) or intraoperative image (such as, e.g., ultrasound or fluoroscopy). Performing the overlay between image and shape data requires a registration between the two modalities.

When two optical shape sensing devices are used in a multi-tether configuration, each of those devices needs to be registered to a same frame of reference. Any error in those registration steps will cause error in the perceived position of the devices. In the case of a guide wire and a catheter, it is a known constraint that during use some portion of the guidewire lies physically within the catheter. If the two shapes are not well registered, the combined output will look as though the guidewire has drifted outside of the catheter. This is clearly not desirable for clinical use.

### SUMMARY

In accordance with the present principles, an optical shape sensing hub includes a longitudinal body forming a cavity configured to receive two or more optical shape sensing (OSS) enabled instruments. One or more mechanical features are disposed within the cavity or on the longitudinal body to maintain the two or more OSS enabled instruments in a fixed geometrical configuration relative to one another such that distally to the longitudinal body the two or more OSS enabled instruments have shape sensed reconstruction data registered therebetween.

A shape sensing system includes a hub comprising a longitudinal body forming a cavity configured to receive two or more optical shape sensing (OSS) enabled instruments, the hub including one or more mechanical features disposed within the cavity or on the longitudinal body to maintain the two or more OSS enabled instruments in a fixed geometrical configuration relative to one another such that distal to the longitudinal body the two or more OSS enabled instruments have shape sensed reconstruction data registered. A shape sensing module is configured to receive, interpret and register optical signals from optical fibers of the two or more OSS enabled instruments to determine shapes of the two or more OSS enabled instruments.

A method for registering two or more an optical shape sensing (OSS) enabled instruments includes providing an optical shape sensing hub comprising a longitudinal body forming a cavity configured to receive two or more optical shape sensing (OSS) enabled instruments, and one or more mechanical features disposed within the cavity or on the longitudinal body to maintain the two or more OSS enabled instruments in a fixed geometrical configuration relative to one another such that distally to the longitudinal body the two or more OSS enabled instruments have shape sensed reconstruction data registered therebetween; generating a hub template of an expected shape of the hub in OSS data; searching measured OSS data to match the hub template to determine a hub position in the OSS data; and determining a registration between the two or more OSS enabled instruments by finding overlap in the OSS data relative to the hub position.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a shape sensing system which employs a hub for registering two or more optical shape sensing (OSS) instruments in accordance with one embodiment;

FIG. 2A is a cross-sectional view of a hub design in accordance with one illustrative embodiment;

FIG. 2B is a cross-sectional view of a hub design showing a distinctive shape for identifying a position of the hub in shape data in accordance with one illustrative embodiment;

FIG. 3 is a cross-sectional view of a hub design where the hub is integrally formed in one of the OSS enabled instruments in accordance with one illustrative embodiment;

FIG. 4 is a flow diagram showing a method for shape-to-shape registration in accordance with an illustrative embodiment;

FIG. 5 shows a plot of a reciprocal of radius of curvature (1/ROC in mm) versus fiber node for three different hub positions on an optical fiber in accordance with an illustrative embodiment;

FIG. 6 is a plot of Kappa versus fiber node showing an example of a hub template employed to identify the hub in OSS data in accordance with an illustrative embodiment;

FIG. 7A shows a plot of absolute value of a difference between outputs of two shape sensing devices (a guidewire and a catheter) versus offset between the two shape sensing devices, a fluctuation in the plot indicates a position of the hub in accordance with an illustrative embodiment;

FIG. 7B is a plot of Kappa versus fiber node showing a region of constant Kappa distal to the hub where the two shape sensing devices are aligned in accordance with an illustrative embodiment;

FIG. 8 is a plot of Kappa versus fiber node showing a region distal to the hub where the two shape sensing devices are registered in accordance with an illustrative embodiment;

FIG. 9A is a diagram showing an OSS enabled guide wire and an OSS enabled catheter shown registered in a pre-operative image of a blood vessel in accordance with an illustrative embodiment;

FIG. 9B is a diagram showing an OSS enabled guide wire and an OSS enabled catheter shown offset from each other in a pre-operative image of a blood vessel;

FIG. 10 is a split-half view of a hub design in accordance with another illustrative embodiment;

FIG. 11 is a split-half view of a hub design in accordance with yet another illustrative embodiment;

FIG. 12 shows three illustrative feature configurations for identifying a hub design is OSS data in accordance with illustrative embodiments; and

FIG. 13 is a split-half view of a hub design showing tracks for three OSS enabled devices in accordance with yet another illustrative embodiment.

### DETAILED DESCRIPTION OF EMBODIMENTS

In accordance with the present principles, a hub device includes a combination of straight and/or curved sections to create a pattern in a shape curvature that is unique and easily identifiable in an optical shape sensing (OSS) system. A position of the hub along a length of a first instrument (e.g., a catheter) can be defined mechanically while a position of the hub along a length of a second instrument (e.g., a guide wire) depends on the amount the second instrument has been inserted through the hub. By detecting the unique curvature pattern (straight-curved-straight, curved-straight-curved, curved-straight, straight-curved, etc., for example) in the shape sensing fiber of the first instrument, it is possible to identify the portion of the second instrument that shares a geometric relationship with the first instrument (e.g., lies within it or next to it, etc.). Then, a registration between those two fibers can be performed using a curvature-based or shape-based implementation.

The hub includes a carefully selected shape for the position where the second instrument, e.g., enters the first instrument. The hub makes it possible to perform real-time shape-to-shape registration between the two instruments. The hub can also be employed for torqueing the instruments. The present principles apply to any integration of optical shape sensing into medical devices where two devices are employed that have a known geometry with respect to each other. In particularly useful embodiments, this applies to guidewires and catheters (either manually and/or robotically controlled), but could be extended to endoscopes, bronchoscopes, etc. and other such applications.

OSS employs light along a multicore optical fiber for device localization and navigation during surgical intervention. One principle involved makes use of distributed strain measurements in the optical fiber using characteristic Rayleigh backscatter or controlled grating patterns (Fiber Bragg Gratings (FBGs)). The shape along the optical fiber begins at a specific point along the sensor, known as the launch or z=0, and the subsequent shape position and orientation are relative to that point.

In multi-tether shape sensing, where multiple instruments are enabled with optical shape sensing, each of these instruments needs to be registered to an imaging frame of reference. Alternatively, if one instrument is registered to the imaging frame of reference then subsequent devices can simply be registered to that first instrument. Registration between devices is known as ‘shape-to-shape’ registration. In a particularly useful embodiment, the present principles provide a hub design for the entry point of, e.g., a guidewire to a catheter, that provides for operator torqueing and handling and shape-to-shape registration between the two or more OSS enabled instruments.

Torqueing is an element of navigation employed for manually steering instruments. For optimal torqueing of an instrument it is beneficial to have an easily graspable handle or feature along the instrument. For example, instrument operators normally gravitate to the location at the guidewire entry point with the catheter as a feature for torqueing and manipulating the catheter. To improve the operator handling and navigation, it is preferable to fit this position along the instrument with a hub design that provides adequate gripping and handling features. Another advantage to mechanically constraining this joint is to improve the optical shape sensing stability. Any joint or transition point along the instrument has the potential to introduce errors or instability in the shape reconstruction. The hub design buffers the fiber from pinching and excessive curvature or tension.

Without shape-to-shape registration each device would need to be independently registered to the image frame of reference. This is not ideal for at least the following reasons. If this registration is performed manually or semi-automatically, it can take additional time to set up each device, and it may take additional fluoroscopy exposure, etc. to register each device to the x-ray field of view. Each registration will have some error, and this will lead to a perceived error between the instruments. Registration may include the launch (or z=0) of both instruments being fixed in space. With shape-to-shape registration, the launch of the guidewire can be floating and all registration may be performed using the hub.

Optical shape sensing can occasionally reconstruct an incorrect shape. This can be due to error in the reconstruction due to proximal shape changes, vibration during the measurement, or pinching of the fiber, among other things. With multiple OSS enabled instruments (e.g., a guidewire and a catheter), shapes may overlap from the point of entry of the catheter to the end of either the catheter or guidewire. By registering the two instruments together, incorrect shapes can be corrected or bad shapes can be removed from the data stream. If both devices have a known fixed launch point, the hub can be employed as an extra control point to filter outliers and compensate for error.

It should be understood that the present invention will be described in terms of medical instruments; however, the teachings of the present invention are much broader and are applicable to any fiber optic shape sensing instruments. In some embodiments, the present principles are employed in tracking or analyzing complex biological or mechanical systems. In particular, the present principles are applicable to internal tracking procedures of biological systems, procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

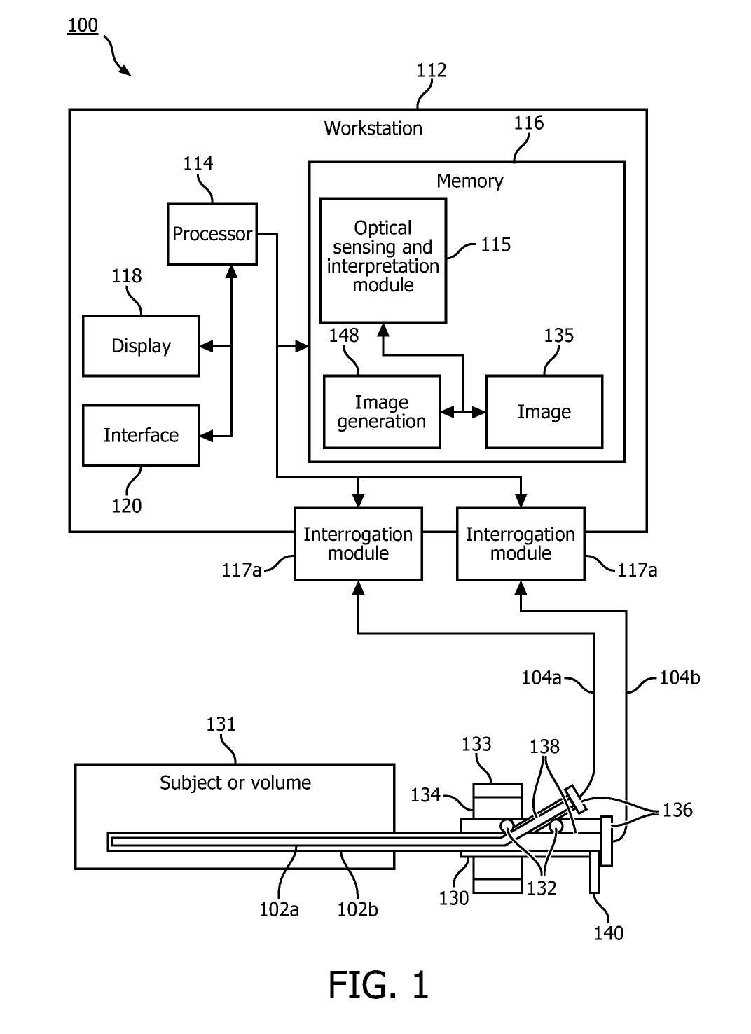

Referring now to the drawings in which like numerals represent the same or similar elements and initially to FIG. 1, a system 100 for optical shape sensing (OSS) using multiple OSS instruments is illustratively shown in accordance with one embodiment. System 100 may include a workstation or console 112 from which a procedure is supervised and/or managed. Workstation 112 preferably includes one or more processors 114 and memory 116 for storing programs and applications. Memory 116 may store an optical sensing module 115 configured to interpret optical feedback signals from a shape sensing device (optical fiber(s)) or system 104. Optical sensing module 115 is configured to use the optical signal feedback (and any other feedback, e.g., electromagnetic (EM) tracking) to reconstruct deformations, deflections and other changes associated with OSS enabled medical devices or instruments 102 and/or their surrounding regions. The medical instruments 102 may include a catheter, a guidewire, a probe, an endoscope, a robot, an electrode, a filter device, a balloon device, or other medical component, etc. Optical sensing module 115 is configured to provide shape-to-shape registration between medical instruments 102. Optical sensing module 115 may include template search or other registration algorithms, filtering or data fitting algorithms, etc. as will be described herein.

In particularly useful embodiments, a plurality of OSS enabled medical instruments 102 are employed together. To ensure registration between these instruments 102, an instrument hub or hub 130 is employed. The hub 130 is depicted to show mechanical features 132 for aligning or registering the instruments 102. The hub 130 may include a polymeric, metal, ceramic or other material suitable for operating or clinical environments. Vibration due to handling of OSS enabled medical instruments 102 is a known limitation to the optical shape sensing performance. This can be mitigated at the hub 130 by employing a vibration-dampening material (e.g., foam or other materials) for manufacturing the hub 130. The hub 130 may include foam portions or may be formed completely from vibration dampening materials.

The hub 130 receives a first shape sensing system 104a from a first optical interrogation module 117a and receives a second shape sensing system 104b from a second optical interrogation module 117b. The hub 130 diverts, within acceptable constraints, the two systems 104a and 104b to register the two systems 104a and 104b. The registration may be achieved by the hub 130 by holding/maintaining the two systems 104a and 104b next to each other, by making the two systems 104a and 104b coincident, by making the two systems 104a and 104b collinear, etc. The hub 130 is configured to physically maintain a geometrical relationship between the two (or more) systems 104a and 104b so that common reconstruction points are shared or can be identified to provide automatic registration between the two (or more) systems 104a and 104b.

In addition, the hub 130 may include other features to permit ergonomic use or to permit improved navigation or manipulation of the two systems 104a and 104b. The features may include a hand grip 133 for ease of use by a clinician. The grip 133 may include a moment arm 134 to enable torqueing of the hub 130 with multiple shape sensing devices 104 therethrough. Other features may include mechanical clamps 136 or other clamping technology to restrict motion of the two systems 104a and 104b. The hub 130 includes tracks 138 for placement of the systems 104a and 104b therein. These tracks 138 are configured to provide a unique and identifiable shape within the shape sensed reconstruction. In addition, these tracks 138 ensure that the minimum bend radius and other physical constraints are maintained by the hub 130.

Depending on the instruments 102a, 102b (collectively referred to as instrument(s) 102) involved, the hub design may be modified to accommodate the structural constraints thereof.

The hub 130 may include adjustment mechanisms or inserts 140 to accommodate different sized or shaped instruments 102 and/or provide accommodation for additional (e.g., more than two) systems 104.

In a particularly useful embodiment, the instruments 102 include a catheter 102b and a guidewire 102a. Each of these two instruments 102a and 102b includes a shape sensing system 104a, and 104b coupled therein or thereto. OSS systems 104a, 104b may also be collectively referred to as system(s) 104.

Each of the shape sensing systems 104a, 104b on instruments 102a, 102b, respectively, include one or more optical fibers (not shown), which are coupled to the instruments 102a, 102b in a set pattern or patterns. The optical fibers connect to the workstation 112 through interrogation modules 117a and 117b, which may be part of the console 112 or may be independent modules. Interrogation modules 117a and 117b send and receive light signals to and from their respective OSS system 104a and 104b. Other cabling may include fiber optics, electrical connections, other instrumentation, etc., as needed to power or operate instruments 102.

Shape sensing systems 104 with fiber optics may be based on fiber optic Bragg grating sensors. A fiber optic Bragg grating (FBG) is a short segment of optical fiber that reflects particular wavelengths of light and transmits all others. This is achieved by adding a periodic variation of the refractive index in the fiber core, which generates a wavelength-specific dielectric mirror. A fiber Bragg grating can therefore be used as an inline optical filter to block certain wavelengths, or as a wavelength-specific reflector.

A fundamental principle behind the operation of a fiber Bragg grating is Fresnel reflection at each of the interfaces where the refractive index is changing. For some wavelengths, the reflected light of the various periods is in phase so that constructive interference exists for reflection and, consequently, destructive interference for transmission. The Bragg wavelength is sensitive to strain as well as to temperature. This means that Bragg gratings can be used as sensing elements in fiber optical sensors. In an FBG sensor, the measurand (e.g., strain) causes a shift in the Bragg wavelength.

One advantage of this technique is that various sensor elements can be distributed over the length of a fiber. Incorporating three or more cores with various sensors (gauges) along the length of a fiber that are embedded in a structure permits a three dimensional form of such a structure to be precisely determined, typically with better than 1 mm accuracy. Along the length of the fiber, at various positions, a multitude of FBG sensors can be located (e.g., 3 or more fiber sensing cores). From the strain measurement of each FBG, the curvature of the structure can be inferred at that position. From the multitude of measured positions, the total three-dimensional form is determined.

As an alternative to fiber-optic Bragg gratings, the inherent backscatter in conventional optical fiber can be exploited. One such approach is to use Rayleigh scatter in standard single-mode communications fiber. Rayleigh scatter occurs as a result of random fluctuations of the index of refraction in the fiber core. These random fluctuations can be modeled as a Bragg grating with a random variation of amplitude and phase along the grating length. By using this effect in three or more cores running within a single length of multi-core fiber, the 3D shape and dynamics of the surface of interest can be followed.

In one embodiment, workstation 112 includes an image generation module 148 configured to receive feedback from the shape sensing system or device 104 and record accumulated position data as to where the sensing device 104 has been within a volume 131 (e.g., a living subject, a mechanical device, ductwork, etc.). An image or image data 135 of the shape sensing instrument(s) 104 within the space or volume 131, generated by the module 148, can be displayed on a display device 118. Workstation 112 includes the display 118 for viewing internal images of a subject (patient) or volume 131 and may include the image 135 as an overlay (e.g., on operative images) or other rendering of visited positions of the sensing systems 104. Display 118 may also permit a user to interact with the workstation 112 and its components and functions, or any other element within the system 100. This is further facilitated by an interface 120 which may include a keyboard, mouse, a joystick, a haptic device, or any other peripheral or control to permit user feedback from and interaction with the workstation 112.

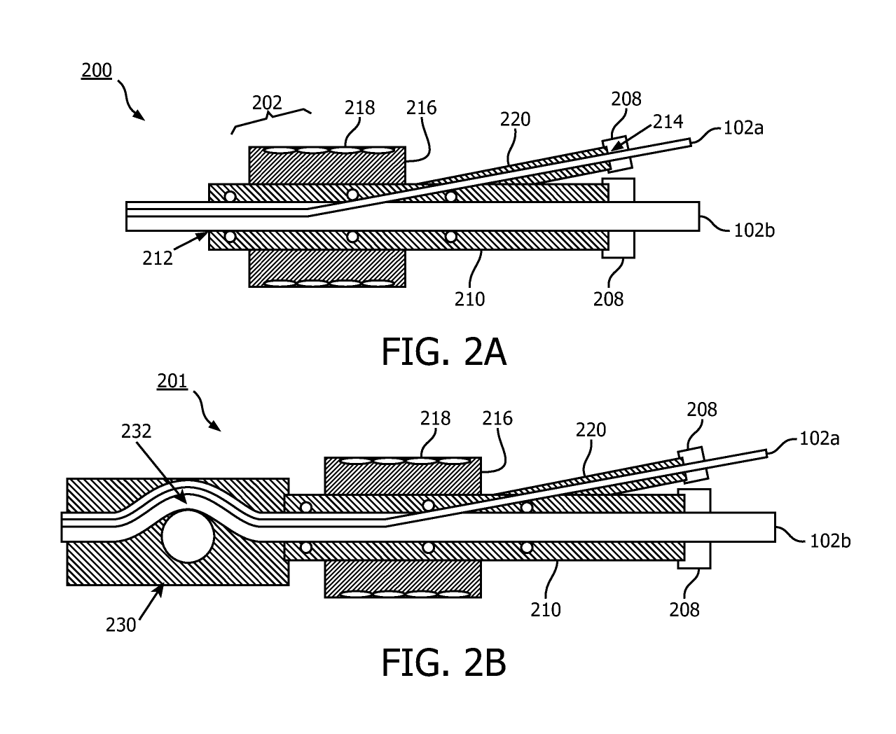

Referring to FIG. 2A, an illustrative hub design 200 is shown in accordance with the present principles. Shape-to-shape registration is provided automatically using the hub design 200. The hub design 200 includes a straight portion 202 that is preferably at least about 60 mm. This straight section 202 may be common to two or more shape sensing systems (104) and may be part of the physical constraints employed for identification and registration of the systems 104.

The hub design 200 preferably includes an ergonomic design suitable for gripping and manipulating the hub system 200 with OSS systems 104 therein. The hub design 200 provides improved torqueing. The size and the shape of the hub design 200 include a natural feature size configured for a hand of a clinician to hold, while also transmitting, or providing a capability to apply, torque directly to a main shaft of a catheter or other instrument 102. The hub design 200 provides for handling a plurality of instruments 102 together. For example, both a catheter and a guidewire are held together and the geometry between these instruments is constrained, e.g., limits the angle of entry between the catheter and the guidewire.

Each instrument 102 may by clamped or otherwise secured in the hub 200 by employing a clamp 208. Clamps 208 may include split half-chucks, include a compression fitting with a thumb screw to apply pressure to an outside diameter of the instrument 102, include a clip, or any other suitable clamping technology. The hub 200 is shown in cross-section and may be made split-half or may include a hollow cavity for inserting the instruments therein.

The hub 200 may include a longitudinal body 210 forming a cavity or track 212 configured to receive at least one OSS enabled instrument 102b. The body 210 includes one or more mechanical features disposed within the cavity or on the longitudinal body 210 to maintain two or more OSS enabled instruments in a fixed geometrical configuration relative to one another such that distally to the longitudinal body 210, the two or more OSS enabled instruments have shape sensed reconstruction data registered therebetween. The mechanical features may include the clamps 208, additional bodies or structures, e.g., an angled channel 220, for guiding at least one OSS enabled instrument (102a), tracks/cavities 212 and/or 214 for maintaining positions of the OSS enabled devices 102, etc. Other mechanical features include radiused tracks (e.g., having a bend radius that exceeds a minimum bend radius of an optical fiber employed for at least one of the two or more OSS enabled instruments), spacers, guides curved surfaces, etc.

In the embodiment depicted, the longitudinal body 210 is configured to receive the OSS enabled device 102b longitudinally therein, and the longitudinal body 210 is also configured with an angled channel 220 to receive the OSS enabled device 102a into the longitudinal body 210 such that when the OSS enabled devices are mounted in the longitudinal body 210 the first and second OSS enabled devices 102a, 102b are positioned coaxially at a distal end portion of the longitudinal body 210.

A radially extending portion 216 extends from the longitudinal body 210 and is configured to provide a torque arm for rotating the hub 200. The radially extending portion 216 may include a grip feature 218 for ergonomic comfort of a user. The radially extending portion 216 may be provided at any convenient or advantageous position along the hub 200 including being combined with other features (e.g., the angled channel 220, etc.).

Referring to FIG. 2B, a hub design 201 preferably includes a detectable registration feature 230. This may include a straight-curved-straight shape (shown ion FIG. 2B) or a curved-straight-curved, curved-straight, etc. The detectable registration feature 230 is configured to provide a shape to the two or more OSS enabled instruments to provide a distinct feature for locating the hub 201 in OSS data. The detectable registration feature 230 includes at least one curved portion 232.

Minimal friction between the instruments 102 (e.g., guide wire) and the hub design 200 or 201 should be maintained. This impacts the material selection for the hub designs 200, 201 and a gentle radius of curvature (ROC) should be employed (for example, >30 mm radius of curvature for a guidewire).

The material for the hub design(s) 200, 201 should include low friction materials, such as, polymeric materials (e.g., polyethylene) or metals (aluminum, stainless, steel), etc. The hub design 200, 201 should not include small radii of curvature bends as they reduce the shape accuracy. A gentle radius of curvature should be employed (e.g., >30 mm ROC).

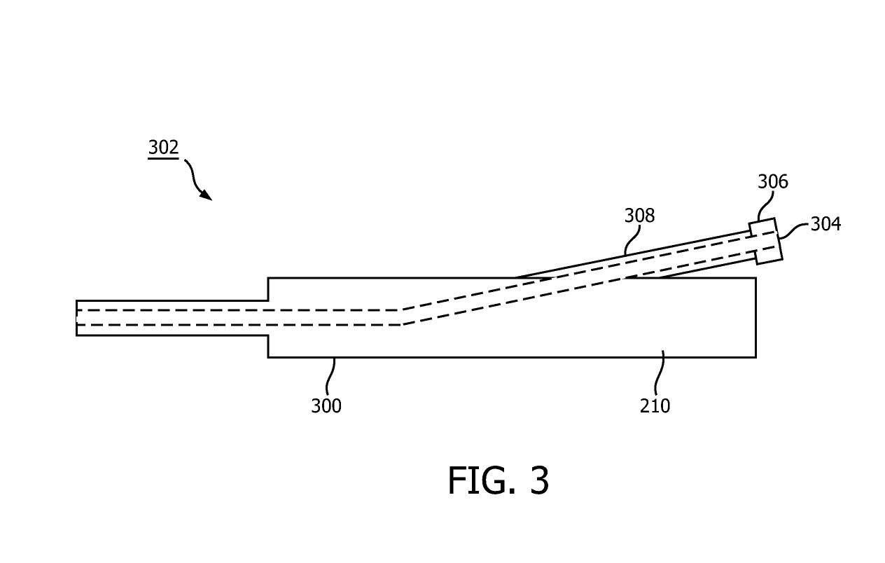

Referring to FIG. 3, a hub 302 is shown integrated into a catheter 300. The longitudinal body 210 is integrally formed as one of the two or more OSS enabled instruments and includes a receiving feature 308 configured to receive at least one other OSS enabled instrument. The hub 302 may be manufactured to be integrally formed with the catheter 300 (or any other device). In one example, the catheter 300 is molded on or with the hub 302, which reduces its form factor and improves its ergonomics. The hub 302 may include insertion points 304 and clamps 306 to mount one or more other OSS enabled devices.

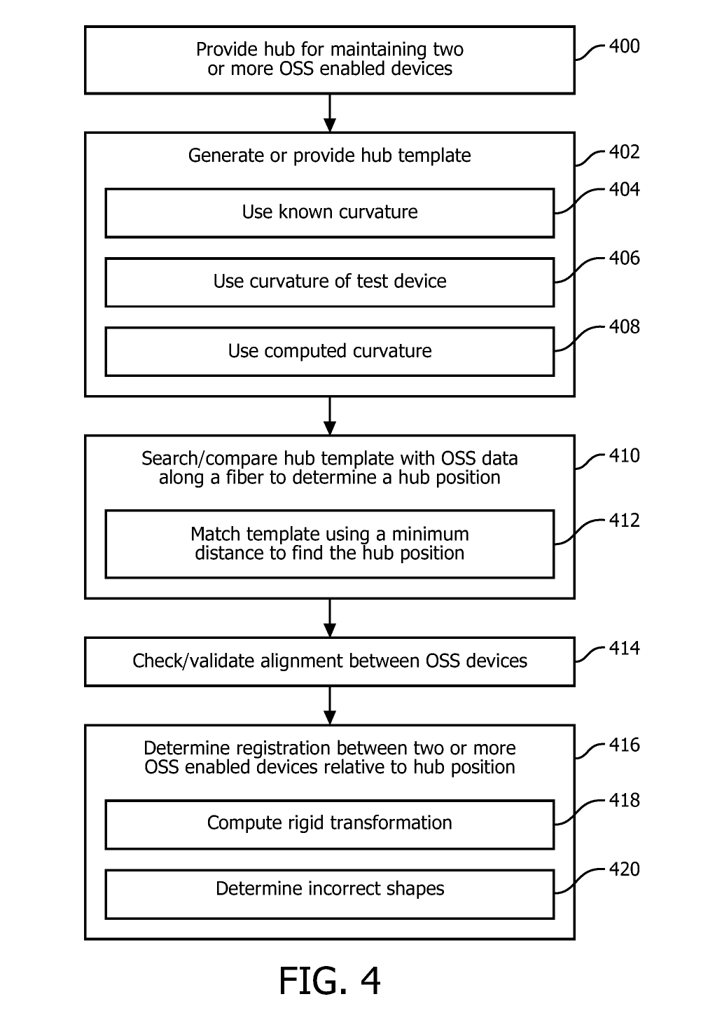

Referring to FIG. 4, a method for shape-to-shape registration is described for registering two or more an optical shape sensing (OSS) enabled instruments using a hub design in accordance with the present principles.

In block 400, an optical shape sensing hub is provided comprising a longitudinal body forming a cavity configured to receive two or more optical shape sensing (OSS) enabled instruments, and one or more mechanical features disposed within the cavity or on the longitudinal body to maintain the two or more OSS enabled instruments in a fixed geometrical configuration relative to one another such that distally to the longitudinal body the two or more OSS enabled instruments have shape sensed reconstruction data registered therebetween.

By employing a unique shape (e.g., a straight-curved-straight shape), the hub provides a curvature/shape for facilitating registration. In block 402, a template of an expected curvature for the hub is generated. The hub template of expected shape is generated in OSS data. The expected shape is preferably an identifiable shape(s) (e.g., straight-curved-straight, etc.). This can be performed in a plurality of ways. In block 404, a known curvature of the hub is employed (e.g., the straight-curved-straight shape). The shape of the hub geometry can be vectorized and assuming the optical fiber takes the shortest available path, the curvatures in the shape are filtered to uniquely identify the hub position.

The fiber and device will take the shortest path, which can be approximated by filtering discrete jumps in expected curvature.

In block 406, the hub template is generated using a curvature of a test device. For example, a device that generates the template shape as an OSS output. The curvature (e.g., the straight-curved-straight shape) may be defined by the test device or devices positioned inside the hub or a specific external test device. In block 408, the hub template may be generated using a computed average from different measurements or other computed shape.

The average or other combinations of different measurements or computations from one or several OSS enabled devices may be employed to locate the hub in the data.

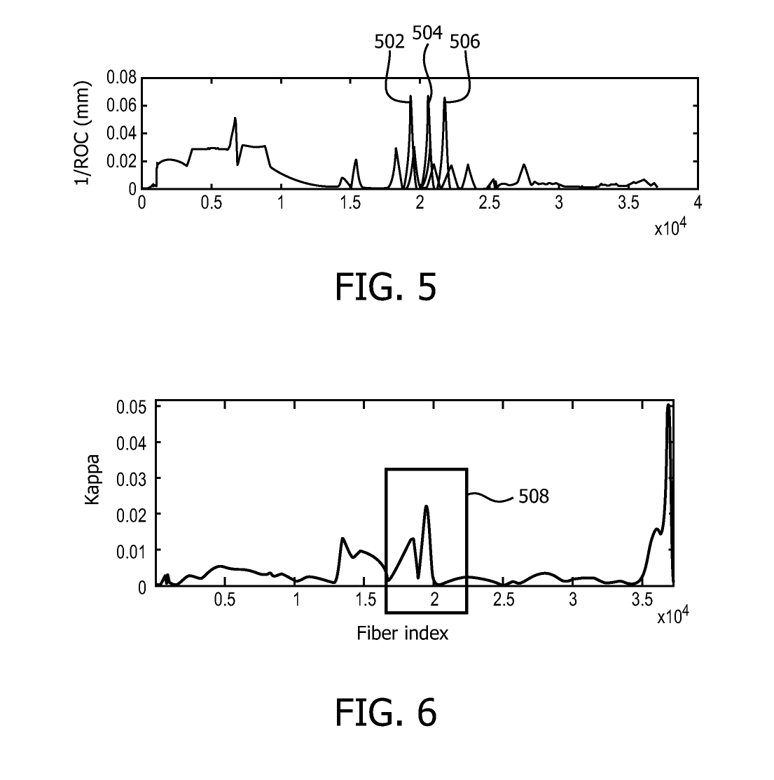

Referring to FIG. 5, a reciprocal of radius of curvature (1/ROC in mm) is plotted versus fiber node for three different hub positions with respect to the fiber indicated by 502, 504 and 506. For the three images a unique pattern can be identified. A known curved shape is indicated by 502, 504 and 506, which is highly unlikely to exist somewhere else along the fiber with the exact same curvature pattern. Completely straight segments are not necessarily unique; however, combined with a curved part, the pattern is longer and therefore more unique. Also, using a straight section improves the need for low friction. In one embodiment, the curvature may be made smaller than a possible bend radius of the instrument. This may be employed for the fiber with the catheter for example. This bend will only occur inside the hub and is therefore uniquely detectable.

The graph of FIG. 5 shows a unique and identifiable pattern for locating the hub in the data using the curved-straight-curved shape. As the hub is translated along the fiber, the position of the hub indicated by 502, 504 and 506 is easily identified by the pattern in the curvature. The identifiable pattern is then employed to match the hub in the data. FIG. 6 shows an example of a template 508 employed to identify the hub in OSS data.

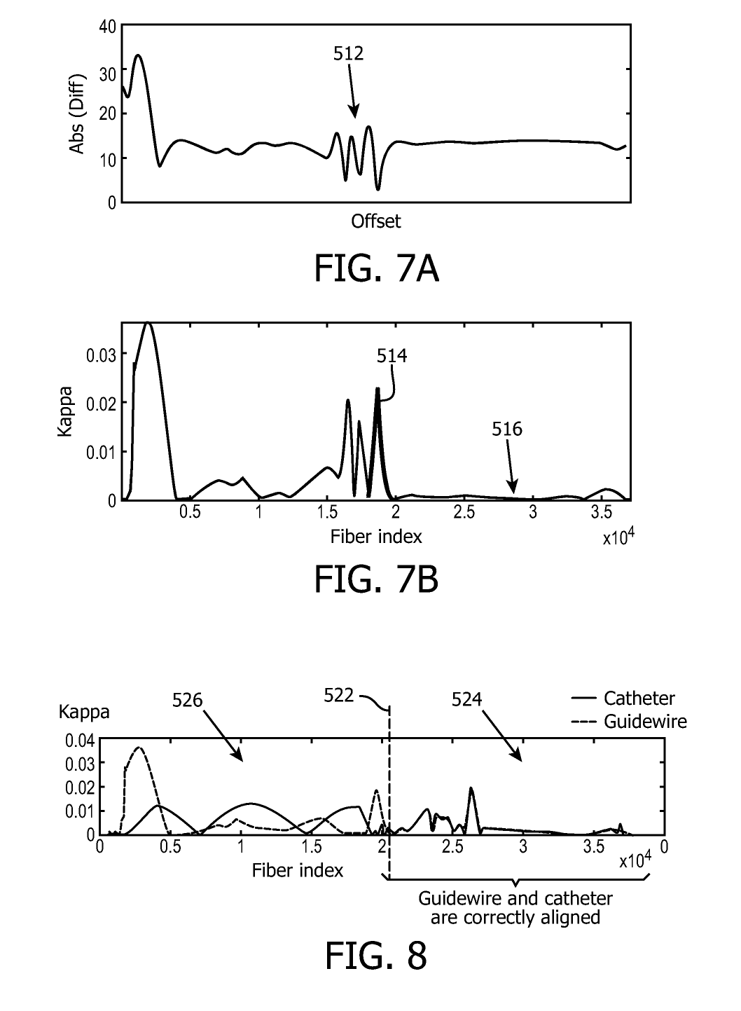

In block 410, for a given shape, compute the curvature along that shape so that the shapes are defined. Measured OSS data is searched (compared) to match the hub template to determine a hub position in the OSS data. In block 412, the hub template may be matched to OSS data to determine a minimum difference to determine the hub position. A template-matching algorithm may be run along the length of the fiber. This can be simply a difference between the template and a selected section of fiber. A minimum value of this matching algorithm will indicate the location of the hub and therefore the relationship between first and second OSS enabled devices. In one example, once the location of the hub is known, the fiber node at which the first OSS enabled device (a guidewire) enters a center lumen of the second OSS enabled device (a catheter) is known. From that point onwards, the two devices will have the same shape until either the guide wire or catheter ends. The algorithm continues:
for a=1: SearchLength-TemplateLength
% compare the template along the length of the instrument (e.g., guidewire)
coeffMatrix(i)=sum(abs(SearchSubject(a:a+TemplateLength−1)−Template));
% sum the difference in Kappa at each node along the template (see FIG. 7A).
   Kappa is a parameter indicating the curvature of the fiber
a=a+1;
end
coeffs=coeffMatrix;
[num, idx]=min(coeffs(:));
Offset=idx.
% the location of the minimum coefficient is the location of the hub along the device (i.e., the point of the guidewire that has been inserted into the hub—this determines the offset between the guidewire and the catheter) (see FIG. 7B).

Referring to FIG. 7A, an absolute value of the difference in curvature between outputs of two shape sensing devices (the guidewire and catheter) is plotted for different offset values between the guidewire and catheter. A local minimum 512 in the graph indicates the position of the hub.

Referring to FIG. 7B, Kappa is plotted against fiber node or index (idx). A region 516 to the right of a fluctuation region 514 (hub position) indicates constant Kappa for the guidewire and catheter distal to the hub.

In block 414, a validation check may be run by checking a correlation between segments of the first and second OSS enabled instruments that are distal to the hub. The validation check or correlation may be provided as an alignment check between OSS data of the two or more OSS enabled instruments. The correlation may be for one or more of curvature, shape or strain.

Referring to FIG. 8, Kappa is plotted against fiber node or index (idx). On a left side 526 of a hub indicated by a dotted line 522, traces of the OSS enabled catheter and guidewire are not the same, as expected as they are not mechanically coupled in that region. However, on a right side 524 of the hub indicated by the dotted line 522, traces of the OSS enabled catheter and guidewire are registered and the traces are in alignment, reflecting that the guidewire is located within the catheter from the hub location onwards, thereby assuming identical curvature.

In block 416, a registration is determined between the two or more OSS enabled instruments by finding overlap in the OSS data relative to the hub position. In block 418, a registration between overlapping portions of the first and second OSS enabled instruments may be computed using a rigid transformation. The rigid transformation may be computed between OSS data of the two or more OSS enabled instruments, and the computation between the two shapes may employ known transformation tools (e.g., Procrustes™ or similar programs). In block 420, incorrect shapes of the instruments (mis-registration of at least one of the OSS enabled instruments) can be determined based upon the registration. These incorrect shapes may be considered outliers and removed from the data set.

It should be understood that the unique shape of the hub design may be employed with one OSS enabled device, with two or more OSS enabled devices using a same unique shape of the hub design or with each OSS enabled device having its own unique shape of the hub design. For example, in addition to using the unique shape of the hub design with the guidewire, it is assumed that the location of the hub along a catheter is also known (e.g., mechanically defined). However, by also adding a simple feature to the catheter path through the hub (straight with a curve), the location of the hub along the catheter could also be extracted using the same technique as is used for the guidewire (e.g., the curvature template matching described above). An additional advantage is that the rotation in relation to the hub would also be known, which cannot be easily derived from a straight path in which the fiber can twist freely.

By knowing the position and the rotation of the hub in the space of the catheter, and by knowing the position of the guidewire in the hub, a registration of one device to the other can be achieved. By uniquely detecting the curved shape (or part of it), a rigid coordinate transformation may be performed using known rigid features of the hub. An actual overlap would not be needed in this case.

A hub with a straight-curved-straight (or other combinations thereof) path where one device enters another can be easily detectable; however, other geometric relationships may be provided that begin at the hub. For example, the two devices may be held side-by-side, or be included in different portions of a same instruments or structure, etc.

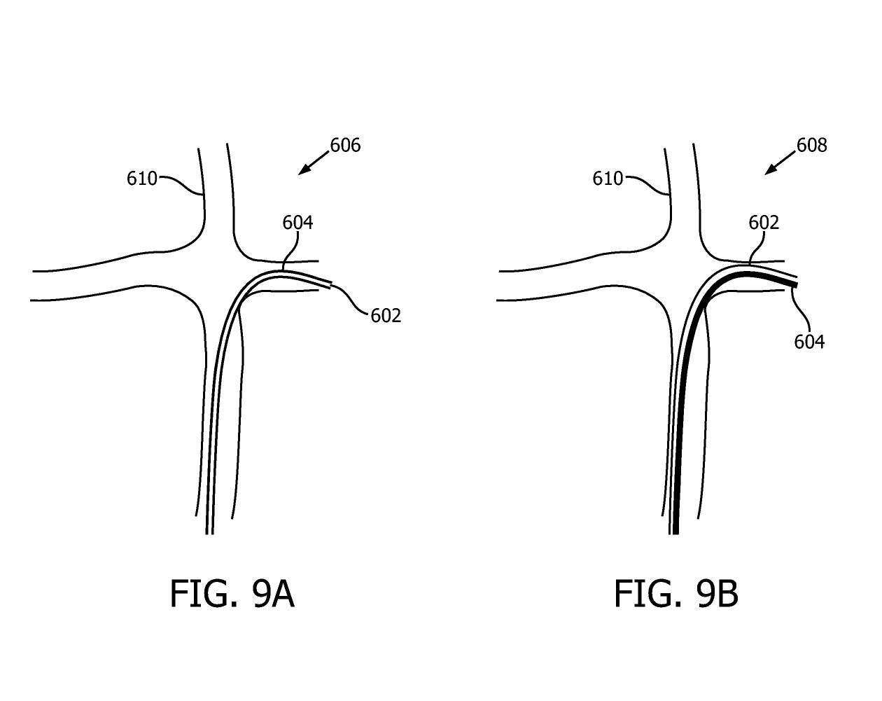

Referring to FIG. 9A and 9B, for meaningful clinical use, the shape-sensed devices need to be registered to an imaging frame of reference (such as, e.g., a pre-operative computed topography (CT) image, a live fluoroscopy image, etc.). An OSS enabled guidewire 602 and an OSS enabled catheter 604 are shown registered to a pre-operative image 606 of a blood vessel 610 in FIG. 9A. For contrast, the OSS enabled guidewire 602 and an OSS enabled catheter 604 are shown poorly registered (offset) in a pre-operative image 608 of the blood vessel 610 in FIG. 9B.

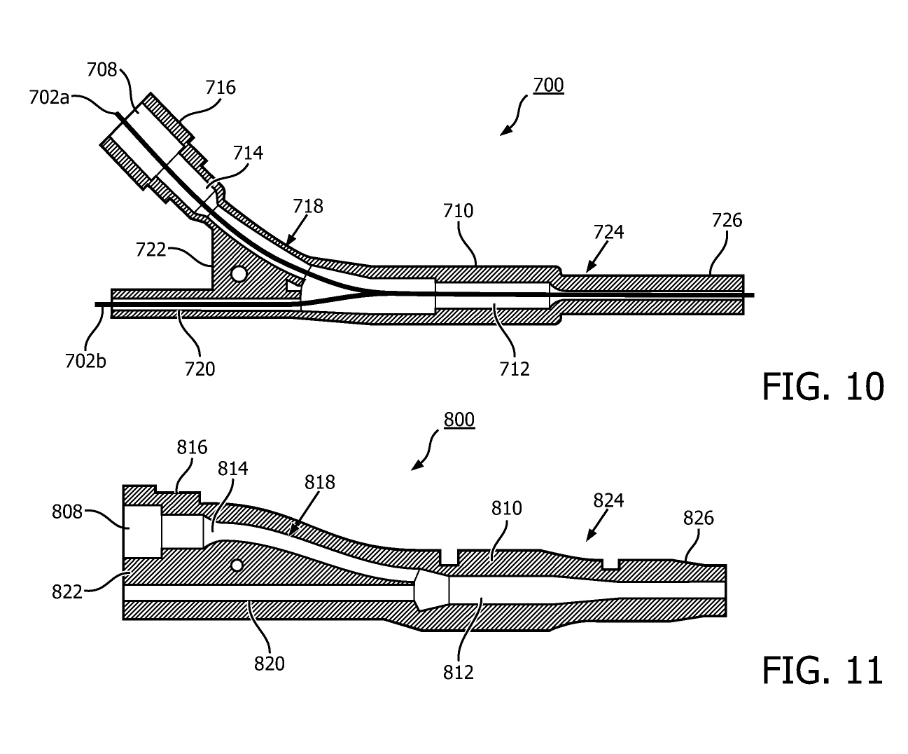

Referring to FIG. 10, the utility of the hub designs described herein may be embodied in a plurality of different configurations. In one embodiment, a hub 700 includes a longitudinal body 710 having Y-shape and shown split-half. A web portion 722 may be employed to provide support or strength to the design of the hub 700 or to provide useful features (a grip, holes for hanging the device, etc.). The longitudinal body 710 includes a straight portion 724 that diverges into a curved portion 718 and an extended portion 720. The curved portion 718, the extended portion 720 or both can contribute to a unique hub shape for locating the hub 700 in OSS data. A first OSS enabled device 702a enters the curved portion 718 through a cavity 708 formed in an end portion 716. The cavity 708 may include a tapered or funnel-like shape 714 to aid in the insertion of the OSS enabled device 702a. A second OSS enabled device 702b enters the extended portion 720 and the OSS enabled devices 702a and 702b are joined in a chamber 712 and exit a distal end portion 726. The configuration of the OSS enabled devices 702a and 702b can be employed to identify the hub 700 in OSS data.

Referring to FIG. 11, a hub 800 includes a longitudinal body 810 having a unitary configuration and split-half. A connection portion 822 may include a solid material employed to provide support or strength to the design of the hub 800 or to provide useful features (holes or pins for connecting its mating half, etc.). The longitudinal body 810 includes a straight portion or track 824 that diverges into a curved portion or track 818 and an extended portion 820. The curved portion 818, the extended portion 820 or both can contribute to a unique hub shape for locating the hub 800 in OSS data. In this embodiment, the curved portion includes multiple bends to contribute to the uniqueness of the shapes.

A first OSS enabled device (not shown) can enter the curved portion 818 through a cavity 808 formed in an end portion 816. The cavity 808 may include a tapered or funnel-like shape 814 to aid in the insertion of the OSS enabled device. A second OSS enabled device (not shown) enters the extended portion 820 and the OSS enabled devices are joined in a chamber 812 and exit a distal end portion 826. The configuration of the OSS enabled devices can be employed to identify the hub 800 in OSS data.

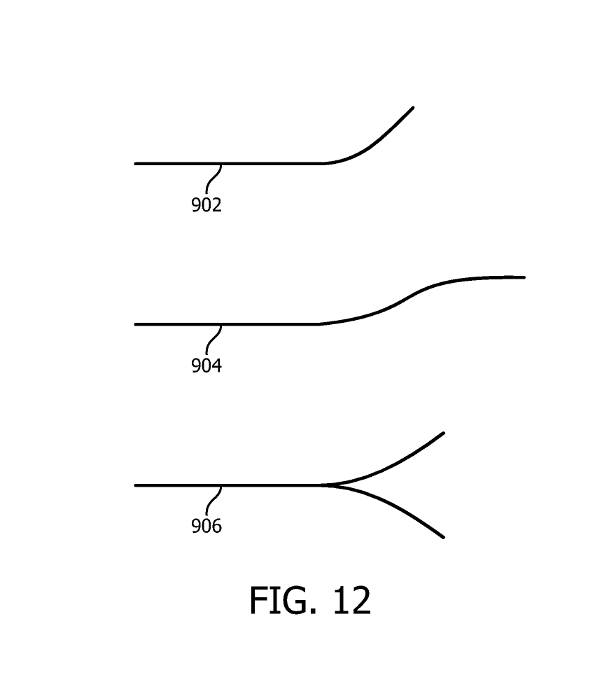

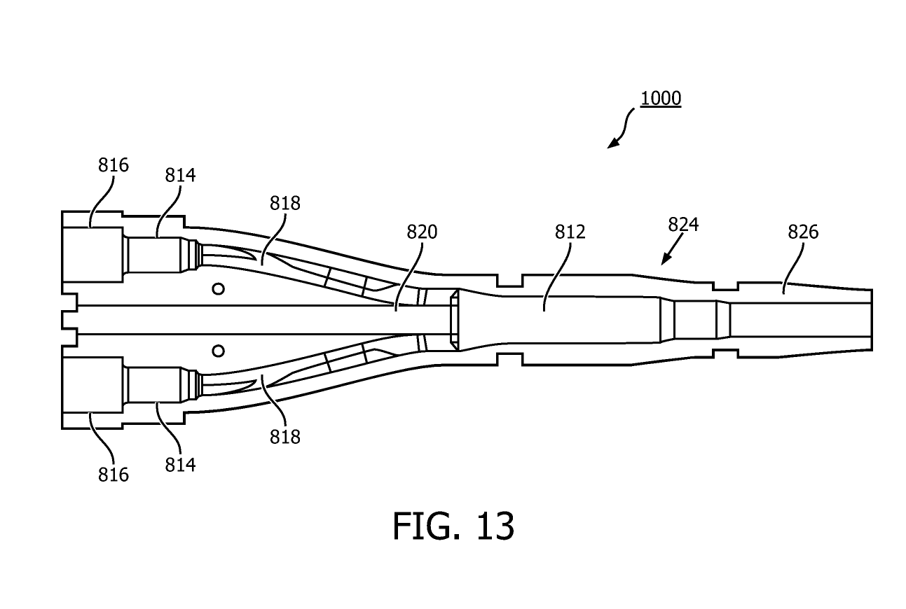

Referring to FIG. 12, three different configurations 902, 904 and 906 of OSS channels for hub designs are illustratively shown. Configuration 902 includes a straight portion and a curved portion. Configuration 904 includes a straight portion and two curved portions. Configuration 906 includes a Y-shaped portion (a curve on each OSS device track) and a common straight portion. It should be understood that other configurations are contemplated and that the configurations depicted in FIG. 12 are for illustrative purposes only.

Referring to FIG. 13, while the present principles depict hub designs configured for two OSS enabled devices, the hub may be configured to accommodate more than two OSS enabled devices. In one embodiment, a hub 1000 includes features, as described with respect to FIG. 11, but includes an additional curved track 818, end portion 816 and entry 814 for receiving an additional OSS enabled device. In one example, the hub 1000 may receive an OSS enabled endoscope (not shown) in track 820. The endoscope may include a working channel. The hub 1000 may also include tracks 818 for one or more OSS enabled catheters, guidewires, etc. The OSS enabled devices in tracks 818 may be passed through the hub 1000 and into the working channel of the endoscope and registered as described above. Other instruments and combinations are also contemplated.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for hub design and methods for optical shape sensing registration (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A hub for optical shape sensing instruments, comprising:
a longitudinal body forming a cavity configured to receive two or more optical shape sensing (OSS) enabled instruments; and
one or more mechanical features disposed within the cavity or on the longitudinal body to maintain the two or more OSS enabled instruments in a fixed geometrical configuration relative to one another such that distally to the longitudinal body the two or more OSS enabled instruments have shape sensed reconstruction data registered therebetween.

2. The hub as recited in claim 1, wherein the longitudinal body is configured to receive a first OSS enabled device longitudinally therein and the longitudinal body is configured with an angled channel to receive a second OSS enabled device into the longitudinal body such that when the first and second OSS enabled devices are mounted in the longitudinal body the first and second OSS enabled devices are aligned at a distal end portion of the longitudinal body.

3. The hub as recited in claim 1, wherein the one or more mechanical features include a track having a bend radius that exceeds a specified minimum bend radius of an optical shape sensing fiber employed for at least one of the two or more OSS enabled instruments.

4. The hub as recited in claim 1, wherein the one or more mechanical features include at least one clamp configured to secure at least one of the two or more OSS enabled instruments.

5. The hub as recited in claim 1, further comprising a radially extending portion from the longitudinal body configured to provide a torque arm for rotating the hub.

6. The hub as recited in claim 5, wherein the radially extending portion includes a grip feature.

7. The hub as recited in claim 1, wherein the longitudinal body is integrally formed as one of the two or more OSS enabled instruments and includes a receiving feature configured to receive at least one other OSS enabled instrument.

8. The hub as recited in claim 1, further comprising a detectable registration feature formed at a distal end portion of the longitudinal body and configured to provide a shape to the two or more OSS enabled instruments to provide a distinct feature for locating the hub in OSS data.

9. The hub as recited in claim 8, wherein the detectable registration feature includes at least one curved portion.

10. A shape sensing system, comprising:
a hub comprising a longitudinal body forming a cavity configured to receive two or more optical shape sensing (OSS) enabled instruments, the hub including one or more mechanical features disposed within the cavity or on the longitudinal body to maintain the two or more OSS enabled instruments in a fixed geometrical configuration relative to one another such that distally to the longitudinal body the two or more OSS enabled instruments have shape sensed reconstruction data registered; and
a shape sensing module configured to receive, interpret and register optical signals from optical fibers of the two or more OSS enabled instruments to determine shapes of the two or more OSS enabled instruments.

11. The system as recited in claim 10, wherein the longitudinal body is configured to receive a first OSS enabled device longitudinally therein and the longitudinal body is configured with an angled channel to receive a second OSS enabled device into the longitudinal body such that when the first and second OSS enabled devices are mounted in the longitudinal body the first and second OSS enabled devices are aligned at a distal end portion of the longitudinal body.

12. The system as recited in claim 10, wherein the one or more mechanical features include:
a track having a bend radius that exceeds a specified minimum bend radius of an optical fiber employed for at least one of the two or more OSS enabled instruments; and
at least one clamp configured to secure at least one of the two or more OSS enabled instruments.

13. The system as recited in claim 10, further comprising a radially extending portion from the longitudinal body configured to provide a torque arm for rotating the hub.

14. The system as recited in claim 10, wherein the longitudinal body is integrally formed as one of the two or more OSS enabled instruments and includes a receiving feature configured to receive at least one other OSS enabled instrument.

15. (canceled)

16. A method for registering two or more an optical shape sensing (OSS) enabled instruments, comprising:
providing an optical shape sensing hub comprising a longitudinal body forming a cavity configured to receive two or more optical shape sensing (OSS) enabled instruments, and one or more mechanical features disposed within the cavity or on the longitudinal body to maintain the two or more OSS enabled instruments in a fixed geometrical configuration relative to one another such that distally to the longitudinal body the two or more OSS enabled instruments have shape sensed reconstruction data registered therebetween;
generating a hub template of an expected shape of the hub in OSS data;
searching measured OSS data to match the hub template to determine a hub position in the OSS data; and
determining a registration between the two or more OSS enabled instruments by finding overlap in the OSS data relative to the hub position.

17. (canceled)

18. (canceled)

19. (canceled)

20. (canceled)

21. (canceled)
